(19) United States
(12) Patent Application Publication
(10) Pub. No.: US 2026/0137933 A1
(43) Pub. Date: May 21, 2026

(54) Localizing Ion-Channel Activation Using Nullifying Waveforms

(71) Applicant: Wisconsin Alumni Research Foundation, Madison, WI (US)

(72) Inventors: James Trevathan, Arena, WI (US); Kip Ludwig, Middleton, WI (US)

(21) Appl. No.: 18/955,228

(22) Filed: Nov. 21, 2024

(51) Int. Cl. A61N 1/36; A61N 1/04

(52) U.S. Cl. CPC A61N 1/36; A61N 1/0428

(57) ABSTRACT

Improved localization of the stimulation of voltage-gated ion channels in ion-channel activation is obtained through the use of nullifying pulses in the vicinity of tissue where stimulation is not desired coordinated with the activating pulse. Non activation by the nullifying pulse is managed variously by control of the timing, voltage, or frequency content of this pulse and/or feedback control.

### STATEMENT REGARDING FEDERALLY SPONSORED RESEARCH OR DEVELOPMENT

This invention was made with government support under NS126376 awarded by the National Institutes of Health. The government has certain rights in the invention.

### BACKGROUND OF THE INVENTION

The present invention relates to the stimulation of tissue having voltage-gated ion channels and in particular to a tissue stimulation device providing improved stimulation localization.

Ion channels provide openings in cell membranes controlling the flow of ions therethrough. The operation of ion channels can be modified by applying an electric field to the ion channels. For example in neurons, such an electrical field can be used to provoke the opening of voltage-gated ion channels leading to action potential initiation (the generation of a nerve signal) in the neuron. Using such techniques to evoke neural activity within a specific neural pathway or nerve is referred to as neuromodulation. Generally, however, modulation of ion channel function can also be used to affect other tissue types including cardiac, skeletal, muscle and epithelial tissues and glial and mast cells.

In the case of neuromodulation, it may be desired to limit the volume of the stimulated tissue, for example, to activate sensory nerves but avoid nearby motor nerves. The volume of tissue can be controlled to some degree by selecting among a variety of stimulating electrode arrangements including monopolar, bipolar, and tripolar configurations. Monopolar stimulation, utilizing a single electrode with a distant reference, produces a more widespread field suitable for broad activation, while bipolar and tripolar stimulation configurations employ two and three electrodes, respectively, to create more localized electric fields. With multiple electrodes, current steering and focusing techniques can be employed to further narrow the stimulation field.

### SUMMARY OF THE INVENTION

The present invention provides a method of localizing ion-channel stimulation by coordinating an activating application of electrical pulses to involved tissue with a nullifying application of electrical pulses to uninvolved tissue. The nullifying pulses alter the electrical and chemical environment of the uninvolved tissue inhibiting ion-channel activation that might otherwise be caused by the activating pulses. The activating and nullifying pulses generally employ different waveform shapes and may have different frequency spectra to take advantage of the sensitivity of the electric potential across the cell membrane to the rate of change of the applied electric field while providing the desired nullifying or stimulating electrical fields. As the term suggests, the nullifying pulses are designed so that they modify the membrane potential to prevent the activation of voltage-gated ion channels in a region of stimulated tissue. Importantly, the nullifying pulses can also be configured to preserve normal ion-channel operation, for example, the transmission of nerve impulses in the non-involved tissue.

The approach of the present invention differs from uncoordinated direct-current (DC) blocking and high-frequency (HF) blocking techniques that utilize continuous or rapidly oscillating electric currents to inhibit ion-channel activity. DC blocking involves the application of a substantially continuous, unidirectional electric current to the tissue making it less responsive to excitatory stimuli but can lead to tissue damage from electrochemical reactions at the electrode-tissue interface. High-frequency blocking exploits the principle that rapid electrical stimulation (for example, above 1 kHz) can nullify neural conduction without causing permanent damage; however, both of these techniques of DC blocking and HF blocking, while preventing activation of the uninvolved tissue, also inhibit normal ion-channel operation of the uninvolved tissue, again, for example, in the case of nerves, interfering with the normal transmission of nerve signals.

In one embodiment, the present invention provides a system for stimulating tissue having voltage-gated ion channels using a first electrode positionable near first tissue having voltage-gated ion channels and a second electrode positionable near second tissue having voltage-gated ion channels. An electrical circuit communicating with these electrodes applies: (a) an activating pulse having a first wave shape to the first electrode adapted to activate the voltage-gated ion channels in the first tissue; and (b) a nullifying pulse having a second waveform shape different from the first waveform shape to the second electrode coordinated in time with the activating pulse and adapted to nullify activation of the voltage-gated ion channels in the second tissue by the combination of the activating and nullifying pulses.

It is thus a feature of at least one embodiment of the invention to use a coordinated nullifying pulse to limit the effect of activating pulses on voltage-gated ion-channel tissues.

The electrical circuit may independently control the current of the activating pulse and nullifying pulse.

It is thus a feature of at least one embodiment of the invention to permit a high degree of flexibility in producing the nullifying pulse through separate electrical control paths.

The average spectral frequency of the activating pulse may be greater than the average spectral frequency of the nullifying pulse.

It is thus a feature of at least one embodiment of the invention to exploit the frequency sensitivity of cells holding the ion channels to minimize ion-channel activation by the nullifying pulse.

The first and second tissue may be nerves and the first and second electrodes may be cuff electrodes fitting around nerves and supporting multiple circumferentially displaced electrodes.

It is thus a feature of at least one embodiment of the invention to provide a system that can be tuned for specific activation through selective application of nullifying and activating signals on different electrodes on a cuff.

The first and second electrodes may be on a common substrate.

It is thus a feature of at least one embodiment of the invention to provide an electrode array allowing for flexible positioning of the nullifying pulses with respect to activating pulses.

The electrical circuit may receive a monitoring signal from a third electrode positioned in communication with the second tissue and operating to control the nullifying pulse to reduce the monitoring signal from the third electrode.

It is thus a feature of at least one embodiment of the invention to permit an automatic optimization of the nullifying pulse in different environments for improved effectiveness while minimizing unintended activation.

The electric circuit may include a multiplexer for selectively connecting a set of different electrodes communicating with the first and second tissue as the first electrode and second electrode to the activating pulse and nullifying pulse and to change the connection to the set of different electrodes. In this way, the electrodes may be selected to optimize stimulation of the first tissue and non-activation of the second tissue.

It is thus a feature of at least one embodiment of the invention to provide post-placement tuning of the localization of electrical stimulation and nullifying pulse locations by switching among different electrodes.

These particular objects and advantages may apply to only some embodiments falling within the claims and thus do not define the scope of the invention.

### BRIEF DESCRIPTION OF THE DRAWINGS

FIG. 1 is a schematic diagram showing electrical connections of a stimulation circuit in one example application using nerve cuffs around the nerve for applying stimulation and nullifying pulses to the electrodes on those cuffs;

FIG. 2 is a timing diagram and spectral plot showing application of a stimulation pulse and nullifying pulse to different nerve fibers in the nerve of FIG. 1;

FIG. 3 is a flowchart of a program executed by the circuit of FIG. 1 in one embodiment; and

FIG. 4 is a figure similar to that of FIG. 2 showing an alternative approach to generating the nullifying pulses.

### DETAILED DESCRIPTION OF THE PREFERRED EMBODIMENT

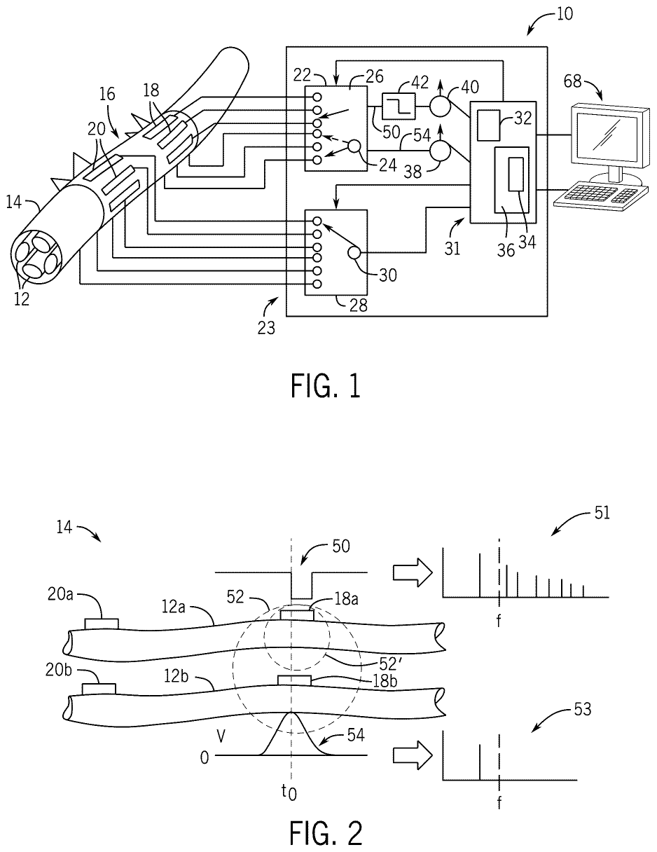

Referring now to FIG. 1, the present invention may provide an ion-channel activation system 10 for applying stimulating and nullifying pulses to voltage-gated ion-channel tissue. As depicted, such tissue may be nerve fibers 12 within a nerve 14 although the present device may be used with a variety of tissue having voltage-gated ion channels including cardiac, skeletal, muscle, and epithelial tissues.

In one embodiment, the nerve 14 may be fit with a cuff electrode 16 wrapping about the nerve 14 and supporting, to be active at an inner surface of the cuff, a set of circumferentially separated modulation electrodes 18. The same cuff electrode 16 or a different cuff electrode may also support a second set of circumferentially separated monitoring electrodes 20, the second set of monitoring electrodes 20 spaced axially from the modulation electrodes 18 along the nerve 14.

Each of the modulation electrodes 18 may be individually connected to a first multiplexer 22 of an electric circuit 23, for example, by a ribbon connector or the like. The multiplexer 22 serves to switchably connect any of the modulation electrodes 18 to one of either a stimulating terminal 24 or nullifying terminal 26. Likewise, each of the monitoring electrodes 20 may be connected to a second multiplexer 28 that may switchably connect any of the monitoring electrodes 20 to a monitoring terminal 30.

The particular switch state of the multiplexers 22 and 28 is controlled by a controller 31, for example, having one or more processors 32 executing a stored program 34 contained in electronic memory 36 as will be described further below.

The stimulating terminal 24 may connect with a switchable voltage or current source 38 also controlled by the controller 31 which may provide a stimulating pulse 50 of voltage to the stimulating terminal 24 at time t₀.

Referring now also to FIG. 2, generally, this stimulating pulse 50 will provide a waveform with sharp rise times or fall times reflecting the fact that transmembrane potential at which voltage-gated ion channels respond is highly dependent on the rate of change of the applied voltage.

In one embodiment, the stimulating pulse 50 may be a rectangular pulse having a duration less than 100 ms or less than 10 ms and a current of less than 100 mA. During normal clinical use, this stimulating pulse 50 will be repeated at a peak frequency from 1 to 300 Hz.

The stimulating pulse 50 as shown and discussed above is a cathodic, unbalanced monophasic, monopolar pulse. More generally, however, it may be a charge-balanced, biphasic pulse, swinging both above and below the tissue ground voltage to capture equal areas between the pulse voltage and ground voltage over the duration of the pulse. In such cases, a user may select either phase as denoting t₀ such as will control the timing of a nullification pulse as discussed below. In some embodiments a separate time t_{0A} and t_{0C} may be associated with each of the anodic and cathodic portions of such a biphasic pulse, respectively, as will be discussed below.

The nullifying terminal 26, in turn, may also communicate with a second switchable voltage or current source 40, for example, through a low-pass filter or slew rate filter 42 or by digital synthesis to produce a band-limited nullifying pulse 54 of voltage to the nullifying terminal 26. This nullifying pulse 54 may be, for example, an anodic, half cycle of a sine wave (from 0-π radians), a sinc pulse or other similar band limited signal. Generally the duration of the nullifying pulse 54 will be from 0.5 ms to the period of the applied stimulation and less than 100 mA. This pulse will be repeated, synchronized to the pulse on stimulating terminal 24 as will be discussed below.

Generally, the nullifying pulse 54 will have a lower average frequency content than the stimulating pulse 50 reflecting the sensitivity of the voltage-gated, ion-channel's rate of change of the electrical field. In some cases, nullifying pulse 54 will have a mean power weighted frequency of less than 10% or less than 50% of the mean frequency of the stimulating pulse 50.

Referring now to FIG. 2, in use, the electric circuit 23 will apply the activating pulse 50 from stimulating terminal 24 to electrode 18a near a nerve fiber 12a intended to have its ion channels activated. The activating pulse 50 will have a rising edge at a time t₀ sufficient, absent the influence of the nullifying pulse 54, to create an electrical field to activate ion channels in an activation region 52 covering both nerve fiber 12a and adjacent nerve fiber 12b. A spectral plot 51 of the pulse 50 shows substantial energy in higher frequencies, for example, above 30 Hz.

Prior to the activation time t₀, the electric circuit 23 may initiate a nullifying pulse 54 from nullifying terminal 26 applied to an electrode 18b spaced apart from electrode 18 a and closer to nerve fiber 12b where ion channel activation is not desired. A spectral plot 53 of the nullifying pulse 54 shows a signal that is largely band limited to have little or no frequency content at higher frequencies, for example, above 30 Hz. This nullifying pulse 54 may be timed so as to reach a peak amplitude at time t₀ (or either of t_{0A} and t_{0C} as selected by user or automatically) and serves to nullify portions of the activation region 52 resulting in a smaller effective activation region 52′while not activating nerve fiber 12b.

While the inventors do not wish to be bound by a particular theory, it is believed that the nullifying pulse 54 alters the electrical environment across the cell membrane of the nerve fiber 12b through the diffusion of ions into that environment to suppress ion and channel activation or shield the nerve fiber 12b from the effects of the pulse 50. The environmental modification, however, is such as not to cause minimal interference with the local propagation of nerve signals. with the local propagation of nerve signals on the nerve fiber 12b.

Referring still to FIG. 2, the effect of the activating pulse 50 will be to generate a nerve impulse passing along the nerve fiber 12a that may be received by a monitoring electrode 20a displaced along the nerve fiber 12a of nerve 14. In contrast, the nullifying pulse 54 does not stimulate the nerve fiber 12b to a degree that produces an off target affect (e.g. causing muscle activation) and in many cases will not produce a nerve impulse to be detected by monitoring electrode 20b or nerve impulses below a predetermined acceptable threshold.

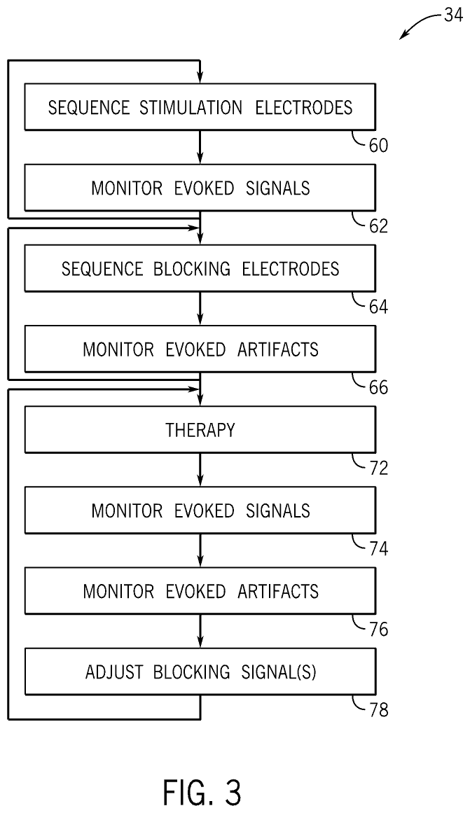

Referring now to FIGS. 1, 2 and 3, the program 34 of the controller 31 depicted in FIG. 1 may be executed by the processor 32, as indicated by process block 60 (FIG. 3) and prior to clinical treatment, to perform a sequencing through the electrodes 18 using the multiplexer 22 to apply, at each sequence step, a stimulating pulse 50. Contemporaneously, the multiplexer 28 may be used to monitor each of the monitoring electrodes 20, per process block 62, to detect any evoked signals on monitoring electrodes 20. This process is used to select a single electrode 18 that best provides a stimulation to a desired nerve fiber 12a while minimizing stimulation of uninvolved nerves 12b and to identify the monitoring electrodes 20 most associated with the nerve fibers 12a or 12b.

At process block 64, a second sequencing is performed through the electrodes 18 (other than the previously selected electrode 18 used for stimulation) to apply nullifying pulses 54 at each sequence step on each of the other electrodes 18, while the previously selected electrode 18 is used for stimulation with stimulating pulse 50. Again, the monitoring electrodes 20 are monitored, at process block 66, to identify one or more electrodes 18 which may receive a nullifying pulse 54 to decrease signals on uninvolved nerve fibers 12b without adversely affecting the evoked signal on the desired nerve fiber 12a. For this purpose multiple electrodes 18 may be selected and used for nullifying pulses 54. In addition, this process may be used to optimize an amplitude and timing of the nullifying pulses which may be the same or different for each electrode 18. In one embodiment, the frequency content of the nullifying pulses 54 may be adjusted from an initially high frequency content while maintaining the charge applied within the nullifying pulse 54. As the high frequency content of the nullifying pulse 54 is decreased, less neural activation should be observed on the monitoring electrodes 20. Once a level of no or minimal neural activation is reached, the activating pulses 50 are introduced to confirm that the nullifying pulse 54 is able to limit off target effect. This may be repeated for different stimulation configurations. The frequency content of the nullification pulse 54 with respect to the stimulating pulse 50 may also be adjusted to accommodate differences which appear to depend on nerve fiber diameter. This may be done iteratively as discussed above or based on measurements of nerve structure. It is believed that the nullification pulse 54 will generally be modified as fiber size decreases to remove more high-frequency content to avoid activation of smaller fibers.

This above described process may be completed semiautomatically (for example, with input from the patient or clinician) or manually by a monitoring of evoked potentials by a clinician using a user terminal 68 connected to the controller 31 indicating the particular electrodes 18 being used for stimulation pulses 50 and nullifying pulses 54 in each sequence and displaying the signals received at the monitoring electrodes 20. Other control inputs that can be used for manually or semiautomatically controlling this process include signals such as measurements of cardiovascular changes, such as heart rate, respiratory rate, EMG. These measurements could be done automatically through sensors (such as measurement electrodes on the device, or semiautomatically via an external device during a programming session with a clinician. In this regard it will be understood that the multiplexer 28 may be operated to allow monitoring of all monitoring electrodes 20 for each sequencing implemented by multiplexer 22.

At process block 72, a therapeutic program may be initiated, using the selected electrode 18 for a stimulating pulse 50 for nerve fiber 12a and one or more other selected electrodes 18 for the nullifying pulse 54 for uninvolved nerve fibers. Interleaved with this therapeutic program, periodically at process block 74, electrodes 20 related to the tissue being treated, and at process block 76 electrodes 20 related to tissue that is not involved in the treatment, may be monitored. This monitoring is then used to adjust the nullifying pulses 54 in timing and amplitude per process block 78 to minimize any signals on electrodes 20b without adversely affecting the stimulation monitored on electrode 20a.

These monitoring measurements may be synchronized with the stimulation pulse 50 to largely reject measurement of incidental nerve signals and may be averaged over multiple cycles.

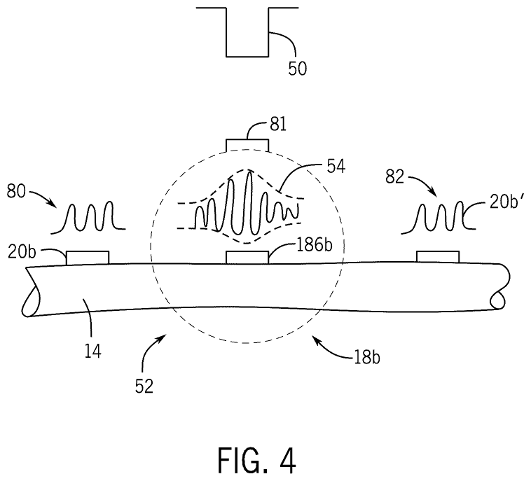

Referring now to FIG. 4, the nullifying pulse 54 may be generated by a pair of electrodes 20b and 20b′ displaced from each other and about the activation region 52, each electrode 20b and 20b′ having a respective applied high-frequency signal 80 and 82, for example, from the circuit 23. The high-frequency signals 80 and 82 have different frequencies to create a beat frequency equal to their frequency difference from an interference when they converge at the activation region 52. The beat frequency will be selected to produce the above described desired frequency of the nullification pulse 54. Generally, the frequency signals 80 and 82 themselves will be too high to recruit sustained neural firing absent this interference.

Note while these electrodes 20b and 20b′ are shown close to the nerve 14, the ability to generate a remote nullifying pulse 54 using an interference generated beat frequency allows electrodes 20b and 20b′, for example, to be placed remotely on the skin. As such, the electrodes 20b and 20b′ may be used an initial evaluation of the ideal location of later, more permanently implanted electrodes.

An additional monitoring electrode 81 may be placed in the activation region 52 to communicate with circuit 24 and monitor the actual electrical field strength in the activation region 52 and more generally in the region of the nerves 14 that are desirably unaffected by the stimulating pulse 50. Signals from this electrode 80 may be used for the automatic implementation of process blocks 64 and 66 described above with respect to FIG. 3 by providing an actual monitoring of the electrical field of the activation region 52.

Certain terminology is used herein for purposes of reference only, and thus is not intended to be limiting., for example,, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. A system for stimulating tissue having voltage-gated ion channels comprising:
a first electrode positionable to interact with first tissue having voltage-gated ion channels;
a second electrode positionable to interact with second tissue having voltage-gated ion channels; and
an electrical circuit applying:
(a) an activating pulse having a first waveform shape to the first electrode adapted to activate the voltage-gated ion channels in the first tissue; and
(b) a nullifying pulse having a second waveform shape different from the first waveform shape to the second electrode, the nullifying pulse at a predetermined synchronization with the activating pulse and adapted to nullify activation of the voltage-gated ion channels in the second tissue by a combination of the activating pulse and nullifying pulse.

2. The system of claim 1 wherein the electrical circuit independently controls a current of the activating pulse and nullifying pulse.

3. The system of claim 1 wherein an average spectral frequency of the activating pulse is greater than the average spectral frequency of the nullifying pulse.

4. The system of claim 1 wherein the nullifying pulses have a lower average frequency content than the stimulating pulses.

5. The system of claim 1 wherein the first and second tissue are nerves and the first and second electrodes are cuff electrodes fitting around nerves and supporting multiple circumferentially displaced electrodes.

6. The system of claim 1 wherein the first and second electrodes are on a common substrate.

7. The system of claim 1 wherein the activating pulse is a biphasic, charge balanced pulse.

8. The system of claim 1 wherein the activating pulse and nullifying pulse have duration of less than 100 ms.

9. The system of claim 1 wherein the electrical circuit receives a monitoring signal from a third electrode positioned in communication with the second tissue and operates to control the nullifying pulse to reduce the monitoring signal from the third electrode.

10. The system of claim 1 wherein the electrical circuit includes a multiplexer for selectively connecting a set of different electrodes communicating with the first and second tissue to change the electrodes of the set operating as the first electrode and second electrode; and
whereby electrodes of the set of different electrodes may be selected to optimize stimulation of the first tissue and nullification of the second tissue.

11. The system of claim 1 wherein the nullifying pulse is a beat frequency produced by an interference between at least two signals having a relative frequency difference equal to a frequency of the nullifying pulse and having an average frequency greater than the frequency of the nullifying pulse.

12. A method of stimulating tissue having voltage-gated ion channels employing a stimulation device having:
a first electrode positionable to interact with first tissue having voltage-gated ion channels;
a second electrode positionable to interact with second tissue having voltage-gated ion channels; and
an electrical circuit applying: an activating pulse having a first waveform shape to the first electrode adapted to activate the voltage-gated ion channels in the first tissue; and a nullifying pulse having a second waveform shape different from the first waveform shape to the second electrode, the nullifying pulse at a predetermined synchronization with the activating pulse and adapted to nullify activation of the voltage-gated ion channels in the second tissue by a combination of the activating pulse and nullifying pulse; the method comprising:
(a) applying an activating pulse to the first tissue to activate voltage-gated ion channels in the first tissue; and
(b) applying a nullifying pulse to the second tissue synchronized to the activating pulse to nullify activation of the voltage-gated ion channels in the second tissue by a combination of the activating pulse and nullifying pulse.

13. The method of claim 12 wherein the electrical circuit independently controls a current of the activating pulse and nullifying pulse.

14. The method of claim 12 wherein an average spectral frequency of the activating pulse is greater than the average spectral frequency of the nullifying pulse.

15. The method of claim 12 wherein the nullifying pulses have a lower average frequency content than the stimulating pulses.

16. The method of claim 12 wherein first and second tissue are nerves and the first and second electrodes are cuff electrodes fitting around nerves and supporting multiple circumferentially displaced electrodes.

17. The method of claim 12 wherein the first and second electrodes are on a common substrate.

18. The method of claim 12 wherein the activating pulse is a biphasic, charge balanced pulse.

19. The method of claim 12 wherein the activating pulse and nullifying pulse have duration of less than 100 ms.

20. The method of claim 12 wherein the electrical circuit receives a monitoring signal from a third electrode positioned in communication with the second tissue and operates to control the nullifying pulse to reduce the monitoring signal from the third electrode.

21. The method of claim 12 wherein the electrical circuit includes a multiplexer for selectively connecting a set of different electrodes communicating with the first and second tissue to change the electrodes operating as the first electrode and second electrode; wherein the method includes:
selecting among the electrodes of the set of different electrodes to optimize stimulation of the first tissue and nullification of the second tissue.

22. The method of claim 12 wherein the second electrode includes a first and second electrode portion wherein the nullifying pulse is expressed as a beat frequency produced in an interference by at least two signals having a relative frequency difference and having an average frequency greater than the nullifying pulse.

23. The method of claim 12 wherein the first and second electrode portions are positioned on a patient's skin.
